(19) United States
(12) Patent Application Publication
(10) Pub. No.: US 2020/0311924 A1
(43) Pub. Date: Oct. 1, 2020

(54) METHOD FOR STORING TOMOGRAPHIC VOLUME DATA, AND TOMOGRAPHY SYSTEM

(71) Applicant: Carl Zeiss Industrielle Messtechnik GmbH, Oberkochen (DE)

(72) Inventors: Jan Schneider, Dresden (DE); Steffen Kunzmann, Dresden (DE); Daniel Flemming, Dresden (DE)

(21) Appl. No.: 16/830,067

(22) Filed: Mar. 25, 2020

(30) Foreign Application Priority Data
Mar. 25, 2019 (DE) 10 2019 204 070.5

(51) Int. Cl. G06T 7/00; G06T 9/00; A61B 6/03

(52) U.S. Cl. CPC G06T 7/0012; G06T 9/004; A61B 6/032; G06T 2207/30004; G06T 2207/10081; G06T 2207/20182; G06T 2200/04; G06T 2207/10016

(57) ABSTRACT

A method for storing tomographic volume data includes segmenting the tomographic volume data into individual slice images, arranging the individual slice images as a sequence of video frames, and compressing and/or decompressing the tomographic volume data by a video compression and/or decompression method). A tomography system includes a compression device configured to compress tomographic volume data by a video compression and/or decompression method, and/or a decompression device configured to decompress the tomographic volume data by the video compression and/or decompression method, the tomographic volume data being segmented into individual slice images, and the individual slice images being arranged as a sequence of video frames. A computer program product includes, and a computer-readable non-transitory storage medium is encoded with instructions that when executed by a processor cause the processor to carry out the method for storing the tomographic volume data.

### CROSS REFERENCE TO RELATED APPLICATIONS

This application claims priority to German patent application DE 10 2019 204 070.5, filed May 25, 2019, the entire content of which is incorporated herein by reference.

### TECHNICAL FIELD

The disclosure relates to a method for storing tomographic volume data, and a tomography system. Further, the disclosure relates to a computer program product and a computer-readable storage medium.

### BACKGROUND

Tomographic information about test objects can be obtained with the aid of imaging methods, for example computed tomography (CT). If a test object is acquired from different radiation directions in each case, a three-dimensional object volume of the test object can be reconstructed from the acquired radiographs. As a rule, associated tomographic volume data includes several hundred megabytes or even a few gigabytes. Such amounts of data present an obstacle, in particular for a transmission via communications network and/or for archiving.

DE 10 2007 018 324 B3 describes an image data acquisition system of an x-ray, CT, or magnetic resonance imaging (MM) device, having an integrated data compression model for a data reduction of acquired image data. Here, acquired image data are already compressed in a data acquisition system that is integrated in the x-ray, CT, or MRI device. As a result, a data rate between the data acquisition system and an image processing and image visualization system can be reduced.

Further, DE 10 2012 204 775 A1 describes a method for reducing and compressing detector raw data of a quantum-counting detector intended for transmission, a data transmission path and a CT system, wherein a bit depth is reduced for compression purposes on the basis of count patterns in the detector raw data.

### SUMMARY

The disclosure addresses the problem of developing a method for storing tomographic volume data and a tomography system, in which a data volume of the tomographic volume data can be reduced in improved fashion.

According to an aspect of the disclosure, the problem is solved by a method for storing tomographic volume data, a tomography system, a computer program product, and a computer-readable storage medium as disclosed herein.

A general concept of the disclosure is the reduction of the data volume of tomographic volume data by virtue of compressing the tomographic volume data by a video (de)compression method. Since the tomographic volume data are available in discrete form in relation to spatial coordinates, the tomographic volume data can be segmented into individual slice images, i.e., slices through the tomographic volume data or through the reconstructed object volume. In the method, the individual slice images of the volume data are each treated like video frames. Together, the individual slice images of the tomographic volume data yield a stack or an ordered sequence, which can be treated like a video film with a plurality of video frames. This procedure is based on the discovery that the object volume or the tomographic volume data can be represented as a stack of individual slice images. Since, as a rule, adjacent slice images—like sequences of video frames that are related in terms of scene—do not differ very greatly from one another, an efficient data reduction can be achieved with the aid of a video (de)compression method. The compressed tomographic volume data can subsequently be stored or archived in a volatile or non-volatile memory, for example for subsequent scrutiny or analysis. As a result of the compression, memory requirements are significantly reduced in relation to memory requirements of the uncompressed tomographic volume data. In order to be able to access the tomographic volume data again following a compression and storage, these are decompressed by the video (de)compression method, with video frames forming individual slices of the tomographic volume data. Subsequently, the tomographic volume data are available again and can be processed and analyzed in customary fashion.

As a result, a data reduction can be achieved that is larger than in the case of a compression of individual radiographs, for example. Further, archiving even comprehensive tomographic volume data is rendered possible.

In particular, a method for storing tomographic volume data, particularly for a tomography system, is made available, wherein the tomographic volume data are compressed and/or decompressed by a video (de)compression method, wherein individual slice images of the tomographic volume data each form video frames in the process.

Further, the tomography system includes a compression device and/or a decompression device, wherein the compression device is configured to compress tomographic volume data by a video (de)compression method and wherein the decompression device is configured to decompress tomographic volume data by the video (de)compression method, wherein individual slice images of the tomographic volume data each form video frames in the process.

Further, the computer program product includes instructions that, upon execution of the program by a computer, a processor, a control module or a programmable hardware component, prompt the latter to carry out the steps of the method in any one of the described exemplary embodiments.

Further, the computer-readable storage medium includes instructions that, upon execution by a computer, a processor, a control module or a programmable hardware component, prompt the latter to carry out the steps of the method in any one of the described exemplary embodiments.

A slice image of the tomographic volume data has a thickness of one voxel of the tomographic volume data or of the reconstructed object volume, in particular. Expressed in simple terms, the stacked slice images together yield the entire object volume or the complete tomographic volume data again.

The term video (de)compression method serves as a common term for a video compression method and a video decompression method. If a compression of tomographic volume data is performed, use is made of the video compression method. By contrast, if a decompression of tomographic volume data is performed, use is made of the corresponding video decompression method.

In particular, the video (de)compression method includes a video codec, i.e., a video (de)compression method that includes or is configured to provide both a coding method and a decoding method.

In particular, the video (de)compression method can include a redundancy reduction. In particular, statistical properties and correlations between spatially adjacent image regions in the slice images used as video frames are exploited in order to describe the video frames using as little data volume as possible in each case (intra-frame coding). Since spatially adjacent image regions in the slice images of the tomographic volume data generally only differ slightly from one another or not at all, this can already achieve a great reduction in the data volume without losing details that are relevant to an analysis of the tomographic volume data.

In particular, the method is carried out by a compression device or by a decompression device. The compression device can be embodied as a combination of hardware and software, for example as program code that is executed on a microcontroller or microprocessor. The compression device receives provided tomographic volume data and compresses the latter by the described method. As a result, the compression device supplies a compressed video which, for example, is output as a video file encoded by way of the corresponding video (de)compression method and is stored in a volatile or non-volatile memory. The decompression device can likewise be embodied as a combination of hardware and software, for example as program code that is executed on a microcontroller or microprocessor. The decompression device receives a provided encoded video file, which contains the compressed video. The decompression device decompresses the compressed video by the described method. As a result, the decompression device supplies video frames which, in the form of slice images, together form the tomographic volume data or the object volume.

One exemplary embodiment provides for the method to be carried out in a computed tomography apparatus. However, in principle, the method can also be used in other tomography systems, for example in a magnetic resonance imaging tomography apparatus or in a nuclear magnetic resonance tomography apparatus.

Alternatively, provision can be made for the method to be realized or have been realized in the form of a computer-implemented method. On the input side, tomographic volume data are retrieved, for example from a memory, and made available to the computer-implemented method. After the computer-implemented method is carried out, the compressed tomographic volume data, on the output side, are output as a compressed video, which is available as a video file encoded by way of the corresponding video (de)compression method, for example, and are stored in a volatile or non-volatile memory. In the case of decompression, the encoded video file is decompressed by the video (de)compression method, the compressed video being received on the input side, for example in the form of an encoded video file. On the output side, the decompressed tomographic volume data are output or provided.

In one exemplary embodiment, provision is made for the video (de)compression method to include inter-frame coding. In inter-frame coding, statistical properties and correlations between temporally adjacent image regions in the slice images of the tomographic volume data or of the video frames are exploited in order to reduce the data volume. This is particularly advantageous since adjacent slice images, which are used as video frames, generally only differ very slightly from one another. Thus, a slice image has great similarity to an adjacent slice image. Thus, an image content of an adjacent slice image can be estimated on the basis of a known slice image. In order to describe differences between the two slice images, it is subsequently sufficient to only ascertain and store an estimation error. Here, a distinction can be made between “P-frames” and “B-frames”. In the case of “P-frames”, a forward estimate is undertaken in a sequence of temporally successive video frames. By contrast, in the case of “B-frames”, there is a bidirectional estimate, implemented on the basis of a preceding and/or a subsequent video frame. By contrast, a so-called “I-frame” denotes an “intra-frame”, which can stand in isolation, independently of the other “frames”. In the “I-frame”, only spatial correlations and similarities of image regions within the “I-frame” are exploited for data reduction. As a rule, the “I-frame” forms a reference image, proceeding from which there can be an estimate of the P- or B-frames. Together, the I-, P-, and B-frames form a group of pictures.

Inter-frame coding can advantageously be used in the case of tomographic volume data since, on account of the continuity of the associated test object, tomographic volume data have no or only very few discontinuous changes from one slice image to the next, as otherwise occur during scene changes in a video sequence, for example. As a result, relatively long series of space-saving P-frames can be coded with few I-frames being used, and so the data volume required to describe the tomographic volume data can be greatly reduced.

In one exemplary embodiment, provision is made for the H.264/MPEG-4 AVC method or the H.265/MPEG-H Part 2 method to be used as the video (de)compression method. Here, the respective methods are used for compressing and decompressing or coding and decoding.

In one exemplary embodiment, provision is made for noise cancellation to be performed before and/or during a compression. This can further reduce the data volume.

In one exemplary embodiment, provision is made for the video (de)compression method to only provide a video file on the output side. In particular, it is possible to dispense with audio data, metadata, and/or subtitles, as are otherwise customary in videos, such that the data volume can be reduced further.

In one exemplary embodiment, provision is made for a container format to be defined or to have been defined for the compressed volume data, wherein the defined container format, in addition to a video file including the tomographic volume data, also includes at least one projection direction information item, a dimension information item, an origin information item, and/or a size of an overall volume in voxels of the tomographic volume data.

In one exemplary embodiment, provision is made for configuration parameters of the video (de)compression method to be selected or to have been selected on the basis of an image quality of the slice images, a resolution of the tomographic volume data, a compression time, a resultant file size, and/or a decompression time. As a result, a resultant data volume following the method being carried out can be adapted to the properties of the output data and/or other targets in a targeted manner. In particular, this can minimize a data volume of the tomographic volume data in a targeted manner, while maintaining the relevant information.

In one exemplary embodiment, provision is made for the container format to include respectively assigned video files for at least two projection directions of the tomographic volume data. Then, provision is made for the tomographic volume data for the at least two projection directions to be compressed individually in each case by the video (de)compression method and for the compressed videos to each be stored in the container. Following the compression, this facilitates the isolated extraction from the compressed video files of video frames or slices of the tomographic volume data derived therefrom for a plurality of projection directions. In particular, it is possible to extract only the video frames or the slices without having to decompress the entire video and restore all of the tomographic volume data. This is particularly advantageous since video frames or slices of different projection directions in even comprehensive tomographic volume data can be compared to one another without great outlay. Therefore, such a comparison can also be implemented on a computer system with little memory (e.g., a desktop computer), which would not permit a simultaneous decompression of the complete tomographic volume data for a plurality of projection directions.

In particular, provision can be made for the container format to include respectively assigned video files for three projection directions of the tomographic volume data. As a result, many applications (comparison of slices from different projection directions, etc.) can already be implemented without, to this end, the tomographic volume data having to be completely decompressed for each projection direction.

Features relating to the configuration of the tomography system arise from the description of configurations of the method. Here, the advantages of the tomography system are respectively the same as in the configurations of the method.

### BRIEF DESCRIPTION OF THE DRAWINGS

The disclosure will now be described with reference to the drawings wherein:

FIG. 1 shows a schematic illustration of the tomography system with a compression device for elucidating a compression of tomographic volume data according to an exemplary embodiment of the disclosure; and

FIG. 2 shows a schematic illustration of a decompression device for elucidating a decompression of tomographic volume data according to an exemplary embodiment of the disclosure.

### DESCRIPTION OF EXEMPLARY EMBODIMENTS

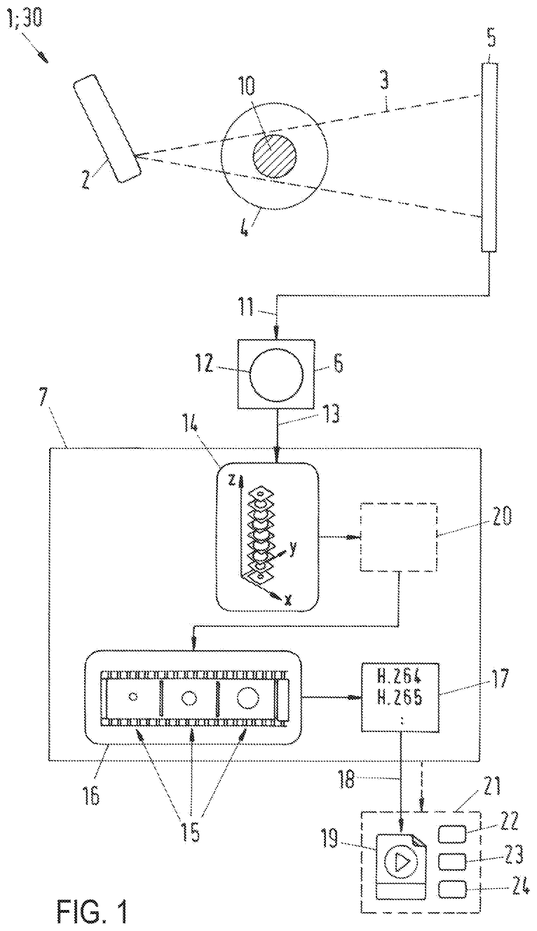

FIG. 1 shows a schematic illustration of the tomography system 1 according to an exemplary embodiment of the disclosure. The tomography system 1 is embodied as a computed tomography apparatus 30 and includes an x-ray source 2 for providing x-ray radiation 3, a rotary table 4, on which a test object 10 can be disposed and rotated, an x-ray detector 5, a control device 6, and a compression device 7.

X-ray radiation 3 is radiated through the test object 10. Radiographs with radiographic data 11 of the test object 10 at different rotary positions of the rotary table 4 are acquired and supplied to the control device 6 by the x-ray detector 5. In particular, this is carried out for a plurality of rotary positions within a complete revolution of the rotary table 4, and so the test object 10 was irradiated and acquired from all rotary directions.

The control device 6 reconstructs an object volume 12 of the test object 10 from the acquired radiographs and provides said object volume in the form of tomographic volume data 13. In particular, this can be implemented by the provision of a digital data packet or a digital file. The tomographic volume data 13 are supplied to the compression device 7.

Using a video (de)compression method 17, the compression device 7 compresses the tomographic volume data 13 provided by the control device 6. To this end, the compression device 7 segments the tomographic volume data 13 into individual slice images 14, the slice images 14 having a thickness corresponding to a voxel dimension in the tomographic volume data 13. The individual slice images 14 are arranged as a sequential, i.e., temporal, sequence of video frames 15 in accordance with their arrangement in the object volume 12 or the tomographic volume data 13. A video 16, i.e., a temporally ordered sequence of video frames 15, is produced from these video frames 15. A timestamp of the individual video frames 15 can be chosen as desired. By way of example, timestamps with the same intervals between temporally adjacent video frames 15 are selected. By way of example, the video 16 is stored as a data packet in a memory of the compression device 7.

The video 16 stored in the memory as a data packet is subsequently compressed by the compression device 7 with the aid of a video (de)compression method 17. As a result, the compression device 7 supplies a compressed video with video data 18, which, for example, is output as a video file 19 encoded by way of the corresponding video (de)compression method 17 and is stored in a transitory or non-transitory memory.

In particular, the video (de)compression method 17 includes inter-frame coding. The video (de)compression method is typically the H.264/MPEG-4 AVC method or the H.265/MPEG-H Part 2 method.

Subsequently, the encoded video file 19 can be archived, for example on a server or in a cloud storage, for a later analysis. Since the data volume of the video file 19 is significantly reduced in comparison with the original tomographic volume data 13, only less memory is required to this end. In particular, tomographic volume data 13 can be archived in their entirety as a result thereof and need not—as is customary—be discarded and deleted following an analysis on account of a lack of storage capacity.

Provision can be made for noise cancellation to be performed before and/or during the compression. This can improve a compression rate when creating the encoded video file 19. A higher compression rate of the encoded video file 19 further reduces the memory requirements. By way of example, noise cancellation can be performed by a noise cancellation device 20.

Further, provision can be made for a container format 21 to be defined or to have been defined for the compressed tomographic volume data 13 by the compression device 7, wherein the defined container format 21, in addition to an encoded video file 19 including the tomographic volume data 13, also includes at least one projection direction information item 22, a dimension information item 23, an origin information item 24, and/or a size of an overall volume in voxels of the tomographic volume data 13.

Further, provision can be made for configuration parameters of the video (de)compression method 17 to be selected or to have been selected on the basis of an image quality of the slice images 14, a resolution of the tomographic volume data 13, a compression time, a resultant file size, and/or a decompression time.

In principle, the method can also be used in other tomography systems 1, such as, for example, in magnetic resonance imaging or in nuclear magnetic resonance imaging.

In principle, the described method need not be carried out in a tomography system 1. The method can also be carried out on any other computing device, for example, a desktop computer, etc., by executing a computer program. In particular, a decompression of the tomographic volume data 13 from the encoded video file 19 can be implemented independently of the tomography system 1.

In order to be able to estimate a capability of the described method, tomographic volume data 13 of a reference data record were compressed by various known compression methods and compared to the method described on the basis of the tomography system 1 or to the video (de)compression method 17.

Tomographic volume data 13 with 788×792×821 pixels or voxels and noise patterns that are typical for computed tomography recordings were examined as a reference data record. The voxels are each encoded in 8-bit grayscales, and so the tomographic volume data 13 include a data volume of 488 megabytes overall.

For comparison purposes, slice images 14 extracted from the tomographic volume data 13 were compressed by known compression methods. The following compression methods were used in the process:
TGA Targa image format with run-length encoding: equal, successive values are replaced by a number of repeating signs and a value of the sign;
ZIP (Standard) ZIP compression by the ZIP module by 7zip 9.20 with standard settings;
PNG Portable Network Graphics; use of the PNGBitmapEncoder of the .NET-Framework with 256 grayscales (8-Bit) as color profile; and
JPEG with a quality setting of 90/100; use of the JPGBitmapEncoder of the .NET-Framework with 256 grayscales (8-Bit) as color profile.

These compression methods were compared to the H.264/MPEG-4 AVC method (“H264”, libx264 codec of the libav library) and the H.265/MPEG-H Part 2 method (“H265”, libx265 codec of the libav library), which are used as the video (de)compression method 17. The following table compares a compression rate, a compression time, and a decompression time of the various methods.

| | | | Compression | Decompression |
|---|---|---|---|---|
| | Compression rate | | time | time |
| TGA | 117% | (573 MB) | 6 s | 1 s |
| ZIP | 23.2% | (113 MB) | 126 s | 3 s |
| PNG | 26.6% | (130 MB) | 10 s | 4 s |
| JPG | 4% | (19.5 MB) | 6 s | 4 s |
| H264 | 0.2% | (0.99 MB) | 2 s | <1 s |
| H265 | 0.08% | (0.38 MB) | 11 s | <1 s |

In the case of the compression rate, the described video (de)compression methods 17 obtain a data reduction that is one to two orders of magnitude better than in the case of the best comparison methods. However, the relevant information is maintained, even in such strongly compressed tomographic volume data 13. In terms of the compression and decompression times, too, the video (de)compression methods 17 used in the method achieve values in the shown example which are of the order of the values of the best compression methods or even exceed the values thereof.

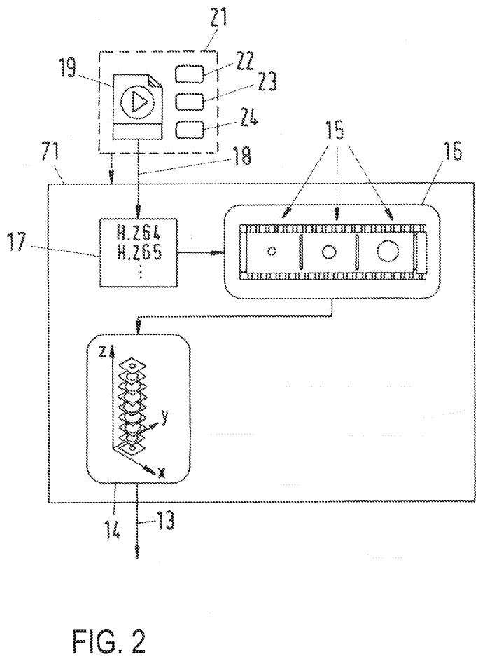

Following the compression, the tomographic volume data 13 can be decompressed again by a decompression device 71 (FIG. 2). To this end, the encoded video file 19 or the compressed video with video data 18 is provided for the decompression device 71, for example by virtue of said encoded video file or said compressed video being retrieved from a volatile or non-volatile memory. The provision can also be implemented in the form of a defined container format 21. The video (de)compression method 17 used during the compression is used during the decompression. In particular, all video frames 15 are completely reconstructed in the process such that each video frame 15 is independent of the other video frames 15 following the decompression and, in each case, independently forms a complete slice image 14. The reconstructed video frames 15 are subsequently arranged as slice images 14 in a layer sequence that corresponds to a temporal sequence of the video frames 15 in the video 16, and the pixels thereof each form the voxels in the tomographic volume data 13. By way of example, a voxel dimension in the decompressed tomographic volume data 13 can be set on the basis of a dimension information item 23 defined in the container format 21. The tomographic volume data 13 are subsequently available again.

The described method for storing tomographic volume data 13 facilitates a great reduction in data and therefore facilitates complete archiving of the tomographic volume data 13 arising therefrom, even in the case of comprehensive measurements, for the purposes of a subsequent analysis. Particularly in the case of network-based communication infrastructures, in which a data memory is not maintained at the same location as the tomography system 1, the tomographic volume data 13 can be stored on a central server and can be retrieved therefrom again, as required, without having to transmit great data volumes over the network.

It is understood that the foregoing description is that of the exemplary embodiments of the disclosure and that various changes and modifications may be made thereto without departing from the spirit and scope of the disclosure as defined in the appended claims.

### LIST OF REFERENCE NUMERALS

1 Tomography system
2 X-ray source
3 X-ray radiation
4 Rotary table
5 X-ray detector
6 Control device
7 Compression device
10 Test object
11 Radiographic data
12 Object volume
13 Tomographic volume data
14 Slice image
15 Video frame
16 Video
17 Video (de)compression method
18 Video data of a compressed video
19 Encoded video file
20 Noise cancellation device
21 Container format
22 Projection direction information item 22
23 Dimension information item
24 Origin information item
30 Computed tomography apparatus
71 Decompression device

What is claimed is:

1. A method for storing tomographic volume data, the method comprising:
segmenting the tomographic volume data into individual slice images;
arranging the individual slice images as a sequence of video frames; and
compressing and/or decompressing the tomographic volume data by a video compression and/or decompression method.

2. The method as claimed in claim 1, wherein the video compression and/or decompression method comprises inter-frame coding.

3. The method as claimed in claim 2, wherein the video compression and/or decompression method is a H.264/MPEG-4 AVC method or a H.265/MPEG-H Part 2 method.

4. The method as claimed in claim 1, further comprising:
performing noise cancellation before and/or during the compressing of the tomographic volume data.

5. The method as claimed in claim 1, further comprising:
defining a container format for the tomographic volume data, wherein the container format includes a video file including the tomographic volume data, at least one projection direction information item, a dimension information item, an origin information item, and/or a size of an overall volume in voxels of the tomographic volume data.

6. The method as claimed in claim 1, further comprising:
selecting configuration parameters of the video compression and/or decompression method based on an image quality of the individual slice images, a resolution of the tomographic volume data, a compression time, a resultant file size, and/or a decompression time.

7. The method as claimed in claim 5, wherein the container format comprises video files respectively assigned for at least two projection directions of the tomographic volume data.

8. The method as claimed in claim 6, wherein the container format comprises video files respectively assigned for at least two projection directions of the tomographic volume data.

9. A tomography system, comprising:
a compression device configured to compress tomographic volume data by a video compression and/or decompression method; and/or
a decompression device configured to decompress the tomographic volume data by the video compression and/or decompression method;
the tomographic volume data being segmented into individual slice images; and
the individual slice images being arranged as a sequence of video frames.

10. The tomography system as claimed in claim 9, wherein the tomography system is a computed tomography apparatus.

11. A computer program product comprising instructions that when executed by at least one of a computer, a processor, a control module, or a programmable hardware component cause the at least one of the computer, the processor, the control module, or the programmable hardware component to carry out the steps of claim 1.

12. A computer-readable non-transitory storage medium encoded with instructions that when executed by at least one of a computer, a processor, a control module, or a programmable hardware component cause the at least one of the computer, the processor, the control module, or the programmable hardware component to carry out the steps of claim 1.
